(19) United States
(12) Patent Application Publication
(10) Pub. No.: US 2005/0196462 A1
(43) Pub. Date: Sep. 8, 2005

(54) Topical formulation containing oxidative reductive potential water solution and method for using same

(75) Inventor: Hojabr Alimi, Santa Rosa, CA (US)

Correspondence Address: LEYDIG VOIT & MAYER, LTD, TWO PRUDENTIAL PLAZA, SUITE 4900, 180 NORTH STETSON AVENUE, CHICAGO, IL, 60601-6780, US

(73) Assignee: Oculus Innovative Sciences, Inc., Petaluma, CA (US)

(21) Appl. No.: 10/916,278

(22) Filed: Aug. 11, 2004

Related U.S. Application Data
(63) Continuation-in-part of application No. 10/862,092, filed on Jun. 4, 2004.
(60) Provisional application No. 60/533,583, filed on Dec. 30, 2003.

(51) Int. Cl. A61K 33/00

(52) U.S. Cl. 424/600

(57) ABSTRACT

A topical formulation containing an oxidative reduction potential (ORP) water solution and a thickening agent that is stable for at least twenty-four hours. The invention also relates to a pharmaceutical dosage form comprising (1) a formulation for topical administration comprising an oxidative reductive potential water solution and a thickening agent and (2) a sealed container, wherein the formulation is stable for at least twenty-four hours. The invention further provides a method for treating or preventing a condition in a patient comprising topically administering to a patient a therapeutically effective amount of a formulation comprising an oxidative reductive potential solution and a thickening agent, wherein the formulation is stable for at least twenty-four hours.

### CROSS-REFERENCE TO RELATED PATENT APPLICATIONS

This application is a continuation-in-part of U.S. patent application Ser. No. 10/862,092, filed on Jun. 4, 2004, which claims the benefit of U.S. Provisional Patent Application 60/533,583, filed on Dec. 30, 2003, which are hereby incorporated by reference.

### FIELD OF THE INVENTION

This invention pertains to formulations for topical administration containing an oxidative reductive potential water solution, such as gels, lotions, creams, ointments, and pastes, methods for the production thereof, and methods for treating a variety of conditions using such formulations.

### BACKGROUND OF THE INVENTION

Oxidative reductive potential (ORP) water, also known as super-oxidized water, can be used as a non-toxic disinfectant to eradicate microorganisms, including bacteria, viruses and spores, in variety of settings. For example, ORP water may be applied in the healthcare and medical device fields to disinfect surfaces and medical equipment. Advantageously, ORP water is environmentally safe and, thus, avoids the need for costly disposal procedures. ORP water also has application in wound care, medical device sterilization, food sterilization, hospitals, consumer households and anti-bioterrorism.

Although ORP water is an effective disinfectant, it has an extremely limited shelf-life, usually only a few hours. As a result of this short lifespan, the production of ORP water must take place in close proximity to where ORP water is to be used as a disinfectant. This means that a healthcare facility, such as a hospital, must purchase, house and maintain the equipment necessary to produce ORP water. Additionally, prior manufacturing techniques have not been able to produce sufficient commercial-scale quantities of ORP water to permit its widespread use as a disinfectant at healthcare facilities.

Accordingly, a need exists for an ORP water that is stable over an extended period of time. A need also exists for a process of preparing commercial-scale quantities of ORP water without additional cost.

ORP water has also been used as a tissue cell growth promoter in patients as described in U.S. patent application Publication 2002/0160053 A1. However, the application of water that quickly loses contact with tissue does not maximize the effectiveness of the treatment. Accordingly, a need exists for compositions containing ORP water that remain in contact with the tissue being treated and that are stable over an extended period of time.

These and other advantages of the invention, as well as additional inventive features, will be apparent from the description of the invention provided herein.

### BRIEF SUMMARY OF THE INVENTION

The invention is directed to a formulation for topical administration comprising an oxidative reductive potential water solution and a thickening agent, wherein the formulation is stable for at least twenty-four hours.

The invention also pertains to a pharmaceutical dosage form comprising (1) a formulation for topical administration comprising an oxidative reductive potential water solution and a thickening agent and (2) a sealed container, wherein the formulation is stable for at least twenty-four hours.

Additionally, the invention is directed to a method for treating a condition in a patient comprising topically administering to a patient a therapeutically effective amount of a formulation comprising an oxidative reductive potential solution and a thickening agent, wherein the formulation is stable for at least about twenty-four hours.

The invention further provides a method for promoting wound healing in a patient comprising applying to a wound a formulation comprising an oxidative reductive potential water solution and a thickening agent, wherein the formulation is administered in an amount sufficient to promote wound healing, and wherein the formulation is stable for at least about twenty-four hours.

Another aspect of the invention includes a method for preventing a condition in a patient comprising topically administering to a patient a therapeutically effective amount of a formulation comprising an oxidative reductive potential water solution and a thickening agent, wherein the formulation is stable for at least about twenty-four hours.

### BRIEF DESCRIPTION OF THE DRAWINGS

FIG. 1 is a schematic diagram of a three chambered electrolysis cell for producing oxidative reductive potential water useful in the present invention.

FIG. 2 is a diagram illustrating a three chambered electrolysis cell for producing oxidative reductive potential water useful in the present invention and the ionic species generated.

FIG. 3 is a schematic flow diagram of the process for producing oxidative reductive potential water useful in the present invention.

### DETAILED DESCRIPTION OF THE INVENTION

According to the present invention, formulations for topical administration comprise an oxidative reductive potential (ORP) water solution and a thickening agent which are prepared to provide enhanced efficacy and stability.

The amount of water present the formulations of the invention is generally from about 10% by weight to about 95% by weight, based on the weight of the formulation. Preferably, the amount of water present is from about 50% by weight to about 90% by weight.

The ORP water solution of the invention may be acidic, neutral or basic, and generally has a pH of from about 1 to about 14. At this pH, the ORP water solution can safely be applied in suitable quantities to hard surfaces without damaging the surfaces or harming objects, such as human skin, that comes into contact with the ORP water solution. Typically, the pH of the ORP water solution is from about 3 to about 8. More preferably, the pH of the ORP water solution is from about 6.4 to about 7.8, and most preferably, the pH is from about 7.4 to about 7.6.

The ORP water solution of the present invention generally has an oxidation-reduction potential of between −1000 millivolts (mV) and +1150 millivolts (mV). This potential is a measure of the tendency (i.e., the potential) of a solution to either accept or transfer electrons that is sensed by a metal electrode and compared with a reference electrode in the same solution. This potential may be measured by standard techniques including, for example, by measuring the electrical potential in millivolts of the ORP water solution relative to standard reference silver/silver chloride electrode. The ORP water generally has a potential between −400 mV and +1300 mV. Preferably, the ORP water solution has a potential between 0 mV and +1250 mV, and more preferably between +500 mV and +1250 mV. Even more preferably, the ORP water of the present invention has a potential of between +800 mV and +1100 mV, and most preferably between +800 mV and +1000 mV.

Various ionic and other species may be present in the ORP water solution of the invention. For example, the ORP water solution may contain chlorine (e.g., free chlorine and bound chlorine), ozone and peroxides (e.g., hydrogen peroxide). The presence of one or more of these species is believed to contribute to the disinfectant ability of the ORP water solution to kill a variety of microorganisms, such as bacteria and fungi, as well as viruses.

Free chlorine typically includes, but is not limited to, hypochlorous acid (HClO), hypochlorite ions (ClO⁻), sodium hypochlorite (NaOCl), chloride ion (Cl⁻), chlorite ions (ClO₂⁻), chlorine dioxide (ClO₂), dissolved chlorine gas (Cl₂), and other radical chlorine species. The ratio of hypochlorous acid to hypochlorite ion is dependent upon pH. At a pH of 7.4, hypochlorous acid levels are from about 25 ppm to about 75 ppm. Temperature also impacts the ratio of the free chlorine component.

Bound chlorine is chlorine in chemical combination with ammonia or organic amines (e.g., chloramines). Bound chlorine is generally present in an amount up to about 20 ppm.

Chlorine, ozone and hydrogen peroxide may present in the ORP water solution of the invention in any suitable amount. The levels of these components may be measured by methods known in the art.

Typically, the total chlorine content, which includes both free chlorine and bound chlorine, is from about 50 parts per million (ppm) to about 200 ppm. Preferably, the total chlorine content is about 80 ppm to about 150 ppm.

The chlorine content may be measured by methods known in the art, such as the DPD colorimeter method (Lamotte Company, Chestertown, Maryland) or other known methods established by the Environmental Protection Agency. In the DPD colorimeter method, a yellow color is formed by the reaction of free chlorine with N,N-diethyl-p-phenylenediamine (DPD) and the intensity is measured with a calibrated calorimeter that provides the output in parts per million. Further addition of potassium iodide turns the solution a pink color to provide the total chlorine value. The amount of bound chlorine present is then determined by subtracting free chlorine from the total chlorine.

Typically, chlorine dioxide is present in an amount of from about 0.01 ppm to about 5 ppm, preferably from about 1.0 ppm to about 3.0 ppm, and more preferably from about 1.0 ppm to about 1.5 ppm. Chlorine dioxide levels may be measured using a modified DPD colorimeter test. Forms of chlorine other than chlorine dioxide are removed by the addition of the amino acid glycine. Chlorine dioxide reacts directly with the DPD reagent to yield a pink color that is measured by a calorimeter machine.

Ozone is generally present in an amount of from about 0.03 ppm to about 0.2 ppm, and preferably from about 0.10 ppm to about 0.16 ppm. Ozone levels may be measured by known methods, such as by a colorimetric method as described in Bader and Hoigne, Water Research, 15, 449-456 (1981). 28 Hydrogen peroxide levels in the ORP water solution are generally in the range of about 0.01 ppm to about 200 ppm, and preferably between about 0.05 ppm and about 100 ppm. More preferably, hydrogen peroxide is present in an amount between about 0.1 ppm and about 40 ppm, and most preferably between about 1 ppm and 4 ppm. Peroxides (e.g., H₂O₂, H₂O₂⁻ and HO₂⁻) are generally present in a concentration of less than 0.12 milliMolar (mM).

The level of the hydrogen peroxide can be measured by electron spin resonance (ESR) spectroscopy. Alternatively, it can be measured by a DPD method as described in Bader and Hoigne, Water Research, 22, 1109-1115 (1988) or any other suitable method known in the art.

The total amount of oxidizing chemical species present in the ORP water solution is in the range of about 2 millimolar (mM) which includes the aforementioned chlorine species, oxygen species, and additional species that may be difficult to measure such as Cl⁻, ClO₃, Cl₂⁻, and ClOₓ. The level of oxidizing chemical species present may also be measured by ESR spectroscopy (using Tempone H as the spin trap molecule).

The ORP water solution of the invention is generally stable for at least twenty-hours, and typically at least two days. More typically, the water solution is stable for at least one week (e.g., one week, two weeks, three weeks, four weeks, etc.), and preferably at least two months. More preferably, the ORP water solution is stable for at least six months after its preparation. Even more preferably, the ORP water solution is stable for at least one year, and most preferably for at least three years.

As used herein, the term stable generally refers to the ability of the ORP water solution remain suitable for its intended use, for example, in decontamination, disinfection, sterilization, anti-microbial cleansing, and wound cleansing, for a specified period of time after its preparation under normal storage conditions (i.e., room temperature).

The ORP water solution of the invention is also stable when stored under accelerated conditions, typically about 30° C. to about 60° C., for at least 90days, and preferably 180 days.

The concentrations of ionic and other species present solution are generally maintained during the shelf-life of the ORP water solution. Typically, the concentrations of free chlorine, chlorine dioxide, ozone and hydrogen peroxides are maintained at about 70% or great from their initial concentration for at least two months after preparation of the ORP water solution. Preferably, these concentrations are maintained at about 80% or greater of their initial concentration for at least two months after preparation of the ORP water solution. More preferably, these concentrations are at about 90% or greater of their initial concentration for at least two months after preparation of the ORP water solution, and most preferably, about 95% or greater.

The stability of the ORP water solution of the invention may be determined based on the reduction in the amount of organisms present in a sample following exposure to the ORP water solution. The measurement of the reduction of organism concentration may be carried out using any suitable organism including bacteria, fungi, yeasts, or viruses. Suitable organisms include, but are not limited to, Escherichia coli, Staphylococcus aureus, Candida albicans, and Bacillus athrophaeus (formerly B. subtilis). The ORP water solution is useful as both a low-level disinfectant capable of a four log (10⁴) reduction in the concentration of live microorganisms and a high-level disinfectant capable of a six log (10⁶) reduction in concentration of live microorganisms.

In one aspect of the invention, the ORP water solution is capable of yielding at least a four log (10⁴) reduction in total organism concentration following exposure for one minute, when measured at least two months after preparation of the solution. Preferably, the ORP water solution is capable of such a reduction of organism concentration when measured at least six months after preparation of the solution. More preferably, the ORP water solution is capable of such a reduction of organism concentration when measured at least one year after preparation of the ORP water solution, and most preferably when measured at least three years after preparation of the ORP water solution.

In another aspect of the invention, the ORP water solution is capable of at least a six log (10⁶) reduction in the concentration of a sample of live microorganisms selected from the group consisting of Escherichia coli, Pseudomonas aeruginosa, Staphylococcus aureus and Candida albicans within one minute of exposure, when measured at least two months after preparation of the ORP water solution. Preferably, the ORP water solution is capable of achieving this reduction of Escherichia coli, Pseudomonas aeruginosa, Staphylococcus aureus or Candida albicans organisms when measured at least six months after preparation, and more preferably at least one year after preparation. Preferably, the ORP water solution is capable of at least a seven log (10⁷) reduction in the concentration of such live microorganism within one minute of exposure, when measured at least two months after preparation.

The ORP water solution of the invention is generally capable of reducing a sample of live microorganisms including, but not limited to, Escherichia coli, Pseudomonas aeruginosa, Staphylococcus aureus and Candida albicans, from an initial concentration of between about 1×10⁶ and about 1×10⁸ organisms/ml to a final concentration of about zero organisms/ml within one minute of exposure, when measured at least two months after preparation of the ORP water solution. This is between a six log (10⁶) and eight log (10⁸) reduction in organism concentration. Preferably, the ORP water solution is capable of achieving this reduction of Escherichia coli, Pseudomonas aeruginosa, Staphylococcus aureus or Candida albicans organisms when measured at least six months after preparation, and more preferably at least one year after preparation.

Alternatively, the ORP water solution is capable of a six log (10⁶ ) reduction in the concentration of a spore suspension of Bacillus athrophaeus spores within about five minutes of exposure, when measured at least two months after preparation of the ORP water solution. Preferably, the ORP water solution is capable of achieving this reduction in the concentration of Bacillus athrophaeus spores when measured at least six months after preparation, and more preferably at least one year after preparation.

The ORP water solution is further capable of a four log (10⁴) reduction in the concentration of a spore suspension of Bacillus athrophaeus spores within about thirty (30) seconds of exposure, when measured at least two months after preparation of the ORP water solution. Preferably, the ORP water solution is capable of achieving this reduction in the concentration of Bacillus athrophaeus spores when measured at least six months after preparation, and more preferably at least one year after preparation.

The ORP water solution is also capable of a six log (10⁶) reduction in the concentration of fungal spores, such as Aspergillis niger spores, within about five to about ten minutes of exposure, when measured at least two months after preparation of the ORP water solution. Preferably, the ORP water solution is capable of achieving this reduction in the concentration of fungal spores when measured at least six months after preparation, and more preferably at least one year after preparation.

In one embodiment, the ORP water solution of the invention comprises hydrogen peroxide (H₂O₂) and one or more chlorine species. Preferably, the chlorine species present is a free chlorine species. The free chlorine species may be selected from the group consisting of hypochlorous acid (HOCl), hypochlorite ions (OCl⁻), sodium hypochlorite (NaOCl), chlorite ions (ClO₂⁻), chloride ion (Cl⁻), chlorine dioxide (CO₂), dissolved chlorine gas (Cl₂), and mixtures thereof

Hydrogen peroxide is present in the ORP water solution generally in the range of about 0.01 ppm to about 200 ppm, and preferably between about 0.05 ppm and about 100 ppm. More preferably, hydrogen peroxide is present in an amount between about 0.1 ppm and about 40 ppm, and most preferably between about 1 ppm and 4 ppm.

The total amount of free chlorine species is generally between about 10 ppm and about 400 ppm, preferably between about 50 ppm and about 200 ppm, and most preferably between about 50 ppm and about 80 ppm. The amount of hypochlorous acid is in the generally between about 15 ppm and about 35 ppm. The amount of sodium hypochlorite is generally in the range of about 25 ppm and about 50 ppm. Chlorine dioxide levels are generally less than about 5 ppm.

The ORP water solution comprising hydrogen peroxide and one or more chlorine species is stable as described herein. Generally, the ORP water solution is stable for at least one week. Preferably, the ORP water solution is stable for at least two months, more preferably, the ORP water solution is stable for at least six months after its preparation. Even more preferably, the ORP water solution is stable for at least one year, and most preferably for at least three years.

The pH of the ORP water solution in this embodiment is generally between about 6 to about 8. Preferably, the pH of the ORP water solution is between about 6.2 and about 7.8, and most preferably between about 7.4 and about 7.6.

While in no way limiting the present invention, it is believed that the control of pH permits a stable ORP water solution in which hydrogen peroxide and chlorine species, such as, by way of example, hypochlorous acid and hypochlorite ions, coexist.

The formulation of the invention preferably includes an ORP water solution comprising anode water and cathode water. Anode water is produced in the anode chamber of the electrolysis cell used in the present invention. Cathode water is produced in the cathode chamber of the electrolysis cell.

Cathode water is generally present in the ORP water solution in an amount of from about 10% by volume to about 90% by volume of the solution. Preferably, cathode water is present in the ORP water solution in an amount of from about 10% by volume to about 50% by volume, more preferably of from about 20%_by volume to about 40% by volume of the solution, and most preferably of from about 20% by volume to about 30% by volume of the solution. Additionally, anode water may be present in the ORP water solution in an amount of from about 50% by volume to about 90% by volume of the solution.

The ORP water solution containing both anode water and cathode water can be acidic, neutral or basic, and generally has a pH of from about 1 to about 14. Typically, the pH of the ORP water solution is from about 3 to about 8. Preferably, the pH is about 6.4 to about 7.8, and more preferably from about 7.4 to about 7.6.

The production of the ORP water solution is carried out by an oxidation-reduction process, also referred to as an electrolytic or redox reaction, in which electrical energy is used to produce chemical change in an aqueous solution. Electrical energy is introduced into and transported through water by the conduction of electrical charge from one point to another in the form of an electrical current. In order for the electrical current to arise and subsist there must be charge carriers in the water, and there must be a force that makes the carriers move. The charge carriers can be electrons, as in the case of metal and semiconductors, or they can be positive and negative ions in the case of solutions.

A reduction reaction occurs at the cathode while an oxidation reaction occurs at the anode in the process for preparing an ORP water solution according to the invention. The specific reductive and oxidative reactions that are believed to occur are described in International Application WO 03/048421 A1.

As used herein, water produced at an anode is referred to as anode water and water produced at a cathode is referred to as cathode water. Anode water contains oxidized species produced from the electrolytic reaction while cathode water contains reduced species from the reaction.

Anode water generally has a low pH typically of from about 1 to about 6.8. Anode water generally contains chlorine in various forms including, for example, chlorine gas, chloride ions, hydrochloric acid and/or hypochlorous acid. Oxygen in various forms is also present including, for example, oxygen gas, peroxides, and/or ozone. Cathode water generally has a high pH typically of from about 7.2 to about 11. Cathode water generally contains hydrogen gas, hydroxyl radicals, and/or sodium ions.

The formulation for topical administration according to the present invention further comprises a thickening agent. Any suitable thickening agent may be used to produce a formulation having the desired viscosity which is generally greater than the ORP water solution alone. The thickening agent utilized is compatible with the ORP water solution and other optional components in the formulation. Suitable thickening agents include, but are not limited to, polymers and hydroxyethylcellulose. Suitable polymers may be homopolymers or copolymers and are optionally crosslinked. Other suitable thickening agents are generally known in art (see, e.g., Handbook of Cosmetic and, Personal Care Additives, 2nd ed., Ashe et al. eds. (2002), and Handbook of Pharmaceutical Excipients, 4th ed., Rowe et al. eds. (2003)).

Preferred thickening agents are acrylic acid-based polymers. More preferably, the thickening agents are high molecular weight, crosslinked, acrylic acid-based polymers. These polymers have the following general structure:

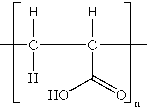

Such polymers are sold under the tradename Carbopol® by Noveon. Carbopol® polymers are generally supplied as rheology modifiers for use thickeners, suspending agents, and stabilizers in a variety of personal care products, pharmaceuticals, and household cleaners. Carbopol® polymers may be used in either solid (e.g., powder) or liquid form.

The acrylic acid-based polymers suitable for use in the invention may be homopolymers or copolymers. Suitable homopolymers may be crosslinked, preferably with allyl sucrose or allylpentaerythritol. Suitable copolymers of acrylic acid are modified by long chain (C₁₀-C₃₀) alkyl acrylates and may be crosslinked, preferably with allylpentaerythritol.

Carbopol® polymers are neutralized in order to achieve maximum viscosity. As supplied, Carbopol® polymers are dry, tightly coiled acidic molecules, held in a coiled structure by hydrogen bonds. Once dispersed in water, or another solvent, they begin to hydrate and partially uncoil. The most common way to achieve maximum thickening from Carbopol® polymers is by converting the acidic polymer into a salt. This is easily achieved by neutralizing with a common base such as sodium hydroxide (NaOH) or triethanolamine (TEA). This neutralization “uncoils” the long chain polymer, swelling the molecule into an effective thickening form.

Suitable thickening agents will yield the desired viscosity for the formulation, as well as other characteristics, such as appearance, shear resistance, ion resistance, and thermal stability. For example, Carbopol® 934 is preferred for a formulation that is either a suspension or emulsion (rather than a clear gel) with a viscosity greater than 3000 centipoise (cps). Carbopol® 974P may alternatively be used for its advantageous bioadhesive properties.

Any suitable amount of a thickening agent is present in the formulation of the invention to yield the desired viscosity for the formulation. Generally, the amount of thickening agent is from about 0.1% by weight to about 50% by weight, based on the weight of the formulation. Preferably, the amount of thickening agent is from about 0.1% to about 10% by weight.

In other terms, the amount of thickening agent based on the volume of the ORP water solution is generally from about 0.1% weight/volume (mg/mL) to about 50% weight/volume (mg/mL). Preferably, the amount of thickening agent is from about 0.1% w/v to about 10% w/v.

The amount of thickening agent generally is from about 0.1 g/250 mL to about 50 mg/250 mL of the ORP water solution. Preferably, the amount of thickening agent present is from about 1 mg/250 mL to about 20 mg/250 mL of the ORP water solution and, most preferably, from about 3 mg/250 mL to about 15 mg/250 mL.

When acrylic acid-based polymers are used at low concentrations, the formulation flows easily with a slippery feel. At higher concentrations, the formulation of the invention has a high viscosity and is pseudoplastic and resistant to flow. When shear force is applied by a mixer or pump, the apparent viscosity is reduced, and the formulation may be pumped.

The formulation of the invention may optionally include a neutralizing agent. Any suitable neutralizing agent may be used to yield the desired pH of the formulation. Suitable neutralizing agents include, for example, sodium hydroxide, triethanolamine, ammonia, potassium hydroxide, L-arginine, AMP-95, Neutrol TE, Tris Amino, Ethomeen, di-isopropanolamine, and tri-isopropanolamine. Other neutralizing agents are generally known in the art (see, e.g., Handbook of Cosmetic and Personal Care Additives, 2nd ed., Ashe et al. eds. (2002), and Handbook of Pharmaceutical Excipients, 4th ed., Rowe et al. eds. (2003)). Suitable neutralizing agents may be either in liquid or solid form.

Preferably, the neutralizer triethanolamine used when the thickening agent is an acrylic acid-based polymer such as Carbopol®. The neutralizing agent converts the formulation into a gel.

Any suitable amount of neutralizing agent may be included in the formulation of the invention. Generally, the amount of neutralizing agent is from about 0.1% by weight to about 50% by weight, based on the weight of the formulation. Preferably, the amount of neutralizing agent is from about 0.1% to about 10% by weight, based on the weight of the formulation. On a volume basis, the amount of neutralizing agent is present in an amount of about 1% to about 50% by volume, based on the volume of the ORP water solution.

When added in liquid form, the neutralizing may be added in an amount of from about 1 mL/250 mL to about 100 mL/250 mL of the ORP water solution. Preferably, the amount of neutralizing agent is from about 10 mL/250 mL to about 90 mg/250 mL of the ORP water solution. Additionally, when in solid form, the neutralizing agent may be added in an amount of from about

The formulation may further contain additional components such as colorants, fragrances, buffers, physiologically acceptable carriers and/or excipients, and the like. Examples of suitable colorants include, but are not limited to, titanium dioxide, iron oxides, carbazole violet, chromium-cobalt-aluminum oxide, 4-Bis[(2-hydroxyethyl)amino]-9,10-anthracenedione bis(2-propenoic)ester copolymers, and the like. Any suitable fragrance can be used.

The formulation of the invention may be prepared by any suitable means. The components of the formulation, such as the ORP water solution and thickening agent, may be mixed together in any manner to yield a homogenous mixture. Preferably, the components are mixed together for several minutes using an electric mixture or other suitable device to ensure uniformity. The components of the formulation are generally mixed from about 400 rpm to about 1000 rpm, preferably from about 500 rpm to about 800 rpm, and more preferably from about 500 rpm to about 600 rpm.

The formulation is mixed for a sufficient period of time to yield a homogenous mixture, generally from about 1 minute to about 10 minutes after all of the components have been combined.

When the thickening agent is in the form of a power, it may first be sieved to break up large agglomerates to allow for the preparation of a homogenous formulation.

A neutralizing agent, such as triethanolamine, may subsequently be added to the formulation containing the ORP water solution and thickening agent. As noted above, the addition of triethanolamine may allow the thickening agent, such as Carbopol®, to uncoil and, thus, yield a formulation having the desired viscosity.

A colorant or fragrance may also be added to the mixture either before or after the thickening agent, such as Carbopol®, is dissolved into the ORP water, but before the neutralization step.

The physical properties of the formulation of the invention are typically the same as those of the ORP water solution present in the formulation. The properties of the ORP water solution remain even after the addition of a thickening agent and optional neutralizing agent. For example, the stability and pH of the ORP water solution itself and the formulation containing the ORP water solution are generally the same. Accordingly, all of the characteristics of the ORP water solution described herein apply to the formulation of the invention.

For example, the formulation of the invention is generally stable for at least twenty-hours, and typically at least two days. More typically, the formulation is stable for at least one week (e.g., one week, two weeks, three weeks, four weeks, etc.), and preferably at least two months. More preferably, the formulation is stable for at least six months after its preparation. Even more preferably, the formulation is stable for at least one year, and most preferably for at least three years.

The pH of the formulation is generally between about 6 to about 8. Preferably, the pH of the formulation is between about 6.2 and about 7.8, and most preferably between about 7.4 and about 7.6.

The formulation of the invention may be used any form suitable for topical administration to a patient. A suitable form includes, but is not limited to, gel, lotion, cream, paste, ointment, and the like, which forms are known in the art (see, e.g., Modem Pharmaceutics, 3rd ed., Banker et al. ed. (1996)). Gels are typically a semisolid emulsion or suspension that has a three-dimensional structure. Preferably, the formulation is in the form of a gel.

Pastes are generally semisolid suspensions that often contain a large portion of solids (e.g., 20% to 50%) dispersed in an aqueous or fatty vehicle. Lotions are typically liquid emulsions containing a water-based vehicle and volatiles (more than 50%) and that have a sufficiently low viscosity (less than 30,000 cps) to be poured. Ointments and creams are generally semisolid emulsions or suspensions that may contain hydrocarbons or polyethylene glycols as part of the carrier along with other volatile components.

When the formulation of the invention is in the form of a gel, the viscosity of the gel is in the range of about 10,000 to about 100,000 centipoise (cps) (e.g., about 15,000 cps, about 20,000 cps, about 25,000 cps, about 30,000 cps, about 35,000 cps, about 40,000 cps, about 45,000 cps, about 50,000 cps, about 55,000 cps, about 60,000 cps, about 65,000 cps, about 70,000 cps, about 75,000 cps, about 80,000 cps, about 85,000 cps, about 90,000 cps, about 95,000 cps, or ranges thereof).

The pH of the gel is typically in the range 6.0 to 8.0. Above this pH, the viscosity of the thickening agent, such as the Carbopol® polymer, may decrease leading to an unsatisfactory topical formulation. Preferably, the pH of the gel is from about 6.4 to about 7.8, and more preferably, from about 7.4 to about 7.6.

Following its preparation, the formulation of the invention may be transferred to a sealed container for distribution and sale to end users such as, for example, health care facilities including hospitals, nursing homes, doctor offices, outpatient surgical centers, dental offices, and the like. The pharmaceutical dosage form according to the present invention comprises the formulation for topical administration as described herein and a sealed container into which the formulation is placed.

Any suitable sealed container may be used that maintains the sterility and stability of the formulation held by the container. The container may be constructed of any material that is compatible with the components of the formulation, for example, the ORP water solution and the thickening agent. The container should be generally non-reactive so that the ions present in the ORP water solution do not react with the container to any appreciable extent.

Preferably, the container is constructed of plastic or glass. The plastic may be rigid so that the container is capable of being stored on a shelf. Alternatively, plastic may be flexible, such as a flexible bag.

Suitable plastics include polypropylene, polyester terephthalate (PET), polyolefin, cycloolefin, polycarbonate, ABS resin, polyethylene, polyvinyl chloride, and mixtures thereof. Preferably, the container comprises polyethylene selected from the group consisting of high-density polyethylene (HDPE), low-density polyethylene (LDPE), and linear low-density polyethylene (LLDPE). Most preferably, the container is high density polyethylene.

The container has an opening to permit dispensing of the formulation for administration to a patient. The container opening may be sealed in any suitable manner. For example, the container may be sealed with a twist-off cap or stopper. Optionally, the opening may be further sealed with a foil layer.

The headspace gas of the sealed container may be air or other suitable gas that does not react with the ORP water solution or other components of the formulation. Suitable headspace gases included nitrogen, oxygen, and mixtures thereof.

The formulation of the invention is suitable for topical administration to a patient, including a human and/or animal, to treat a variety of conditions. Specifically, the formulation may be applied to animals (e.g., mice, rats, pigs, cows, horses, dogs, cats, rabbits, guinea pigs, hamsters, birds) and humans. Topical administration includes application to the skin as well as oral, intranasal, intrabronchial, and rectal routes of administration.

In another embodiment, the invention is directed to a method for treating a condition in a patient by topically administering a formulation comprising an ORP water solution and a thickening agent.

Conditions in a patient that may be treated according to the invention include, for example, the following: surgical/open wound cleansing agent; skin pathogen disinfection (e.g., for bacteria, mycoplasmas, virus, fungi, prions); wound disinfection (e.g., battle wounds); wound healing promotion; burn healing promotion; treatment of skin fungi; psoriasis; athlete's foot; ear infections (e.g., swimmer's ear); traumatic wounds; acute, subchronic and chronic infections (e.g. diabetic foot infections being an example of the latter), pressure ulcers, derma-abrasion, debrided wounds, laser re-surfacing, donor sites/grafts, exuding partial and full thickness wounds, superficial injuries (lacerations, cuts, abrasions, minor skin irritations) and other medical applications on or in the human or animal body. Ulcers treated according to the invention may or may not have abscesses or necrotic tissue present.

Additionally, the invention is directed to a method for promoting wound healing in a patient by applying to a wound a formulation comprising an oxidative reductive potential water solution and a thickening agent. The wound to be treated may be caused by any surgery, ulcer or other means. Ulcers that may be treated include, for example, diabetic foot ulcers.

The invention further relates to a method for preventing a condition in a patient by topically administering a formulation comprising an ORP water solution and a thickening agent. For example, the formulation (e.g., in the form of a gel) can be used as a barrier on open wounds to prevent infection. Specifically, the formulation (e.g., in the form of a gel) can be applied to the surface of a wound, such as a foot ulceration in a diabetic, who is prone to neurological and vascular complications. The formulation applied thusly can provide a barrier to infection, since these wounds are the principal portal for infection for diabetic patients.

The formulation may be used to prevent sexually transmitted diseases in a patient including, for example, infections. Such infections that may be prevented include herpes, human immunodeficiency virus (HIV) and vaginal infections. When the formulation is in the form of a gel, it may be used as a spermicide.

While in no way limited the present invention, it is believed that the ORP water solution eradicates the bacteria with which it contacts as well as destroying the bacterial cellular components including proteins and DNA.

The formulation of the invention may be used or applied in a therapeutically effective amount to provide the desired therapeutic effect on bacteria, viruses, and/or germs. As used herein, a therapeutically effective amount refers to an amount of the formulation that results in an improvement of the condition being treated or to be prevented. For example, when used to treat an infection, a therapeutically effective amount of the formulation reduces the extent of the infection and/or prevents further infection. As is appreciated by one skilled in the art, the efficacy of the formulation of the invention resulting from administering the formulation may be short-term (i.e., a few days) and/or long-term (e.g., months).

The formulation may further be applied over a sufficient period of time, for example, one two, several days, one week, or several weeks, until the desired effect on the patient is observed.

The formulation may be applied in any suitable manner. For example, a quantity of the formulation may be applied to the surface of the patient to be treated and then evenly spread using the patient's own fingers. Alternatively, a health care provider may apply the formulation to the patient's tissue. A suitable implement, for example, a disposable wipe or cloth, may be used to apply the formulation.

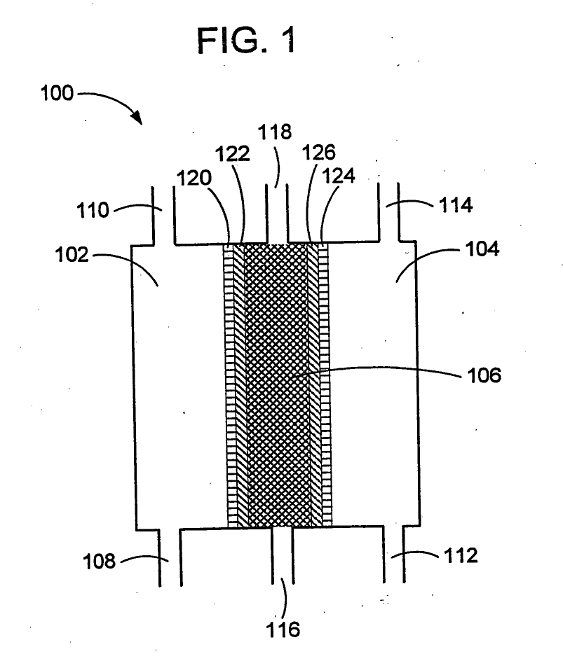

The ORP water solution used in the present invention may be prepared by any suitable means. Preferably, the ORP water solution is produced using at least one electrolysis cell comprising an anode chamber, cathode chamber and salt solution chamber located between the anode and cathode chambers, wherein the ORP water solution comprises anode water and cathode water. A diagram of a typical three chamber electrolysis cell useful in the invention is shown in FIG. 1.

The electrolysis cell 100 has an anode chamber 102, cathode chamber 104 and salt solution chamber 106. The salt solution chamber is located between the anode chamber 102 and cathode chamber 104. The anode chamber 102 has an inlet 108 and outlet 110 to permit the flow of water through the anode chamber 100. The cathode chamber 104 similarly has an inlet 112 and outlet 114 to permit the flow of water through the cathode chamber 104. The salt solution chamber 106 has an inlet 116 and outlet 118. The electrolysis cell 100 preferably includes a housing to hold all of the components together.

The anode chamber 102 is separated from the salt solution chamber by an anode electrode 120 and an anion ion exchange membrane 122. The anode electrode 120 may be positioned adjacent to the anode chamber 102 with the membrane 122 located between the anode electrode 120 and the salt solution chamber 106. Alternatively, the membrane 122 may be positioned adjacent to the anode chamber 102 with the anode electrode 120 located between the membrane 122 and the salt solution chamber 106.

The cathode chamber 104 is separated from the salt solution chamber by a cathode electrode 124 and a cathode ion exchange membrane 126. The cathode electrode 124 may be positioned-adjacent to the cathode chamber 104 with the membrane 126 located between the cathode electrode 124 and the salt solution chamber 106. Alternatively, the membrane 126 may be positioned adjacent to the cathode chamber 104 with the cathode electrode 124 located between the membrane 126 and the salt solution chamber 106.

The electrodes are generally constructed of metal to permit a voltage potential to be applied between the anode chamber and cathode chamber. The metal electrodes are generally planar and have similar dimensions and cross-sectional surface area to that of the ion exchange membranes. The electrodes are configured to expose a substantial portion of the surface of the ion exchange members to the water in their respective anode chamber and cathode chamber. This permits the migration of ionic species between the salt solution chamber, anode chamber and cathode chamber. Preferably, the electrodes have a plurality of passages or apertures evenly spaced across the surface of the electrodes.

A source of electrical potential is connected to the anode electrode 120 and cathode electrode 124 so as to induce an oxidation reaction in the anode chamber 102 and a reduction reaction in the cathode chamber 104.

The ion exchange membranes 122 and 126 used in the electrolysis cell 100 may be constructed of any suitable material to permit the exchange of ions between the salt solution chamber 106 and the anode chamber 102 such as chloride ions (Cl⁻) and between the salt solution salt solution chamber 106 and the cathode chamber 104 such as sodium ions (Na⁺). The anode ion exchange membrane 122 and cathode ion exchange membrane 126 may be made of the same or different material of construction. Preferably, the anode ion exchange membrane comprises a fluorinated polymer. Suitable fluorinated polymers include, for example, perfluorosulfonic acid polymers and copolymers such as perfluorosulfonic acid/PTFE copolymers and perfluorosulfonic acid/TFE copolymers. The ion exchange membrane may be constructed of a single layer of material or multiple layers.

The source of the water for the anode chamber 102 and cathode chamber 104 of the electrolysis cell 100 may be any suitable water supply. The water may be from a municipal water supply or alternatively pretreated prior to use in the electrolysis cell. Preferably, the pretreated water is selected from the group consisting of softened water, purified water, distilled water, and deionized water. More preferably, the pretreated water source is ultrapure water obtained using reverse osmosis purification equipment.

The salt water solution for use in the salt water chamber 106 may be any aqueous salt solution that contains suitable ionic species to produce the ORP water solution. Preferably, the salt water solution is an aqueous sodium chloride (NaCl) salt solution, also commonly referred to as a saline solution. Other suitable salt solutions include other chloride salts such as potassium chloride, ammonium chloride and magnesium chloride as well as other halogen salts such as potassium and bromine salts. The salt solution may contain a mixture of salts.

The salt solution may have any suitable concentration. The salt solution may be saturated or concentrated. Preferably, the salt solution is a saturated sodium chloride solution.

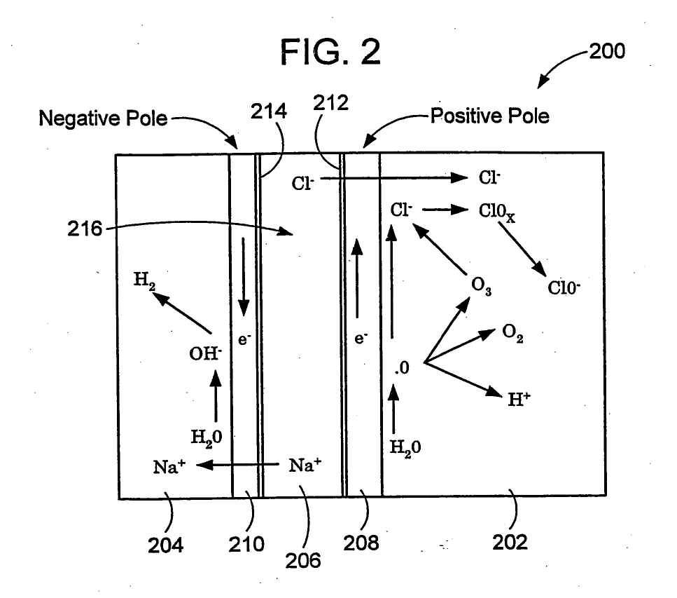

The various ionic species produced in the three chambered electrolysis cell useful in the invention are illustrated in FIG. 2. The three chambered electrolysis cell 200 includes an anode chamber 202, cathode chamber 204, and a salt solution chamber 206. Upon application of a suitable electrical current to the anode 208 and cathode 210, the ions present in the salt solution flowing through the salt solution chamber 206 migrate through the anode ion exchange membrane 212 and cathode ion exchange membrane 214 into the water flowing through the anode chamber 202 and cathode chamber 204, respectively.

Positive ions migrate from the salt solution 216 flowing through the salt solution chamber 206 to the cathode water 218 flowing through the cathode chamber 204. Negative ions migrate from the salt solution 216 flowing through the salt solution chamber 206 to the anode water 220 flowing through the anode chamber 202.

Preferably, the salt solution 216 is aqueous sodium chloride (NaCl) that contains both sodium ions (Na⁺) and chloride ions (Cl⁻) ions. Positive Na⁺ ions migrate from the salt solution 216 to the cathode water 218. Negative Cl-ions migrate from the salt solution 216 to the anode water 220.

The sodium ions and chloride ions may undergo further reaction in the anode chamber 202 and cathode chamber 204. For example, chloride ions can react with various oxygen ions and other species (e.g., oxygen free radicals, O₂, O₃) present in the anode water 220 to produce ClOn- and ClO⁻. Other reactions may also take place in the anode chamber 202 including the formation of oxygen free radicals, hydrogen ions (H⁺), oxygen (as O₂), ozone (O₃), and peroxides. In the cathode chamber 204, hydrogen gas (H₂), sodium hydroxide (NaOH), hydroxide ions (OH⁻), ClOn-ions, and other radicals may be formed.

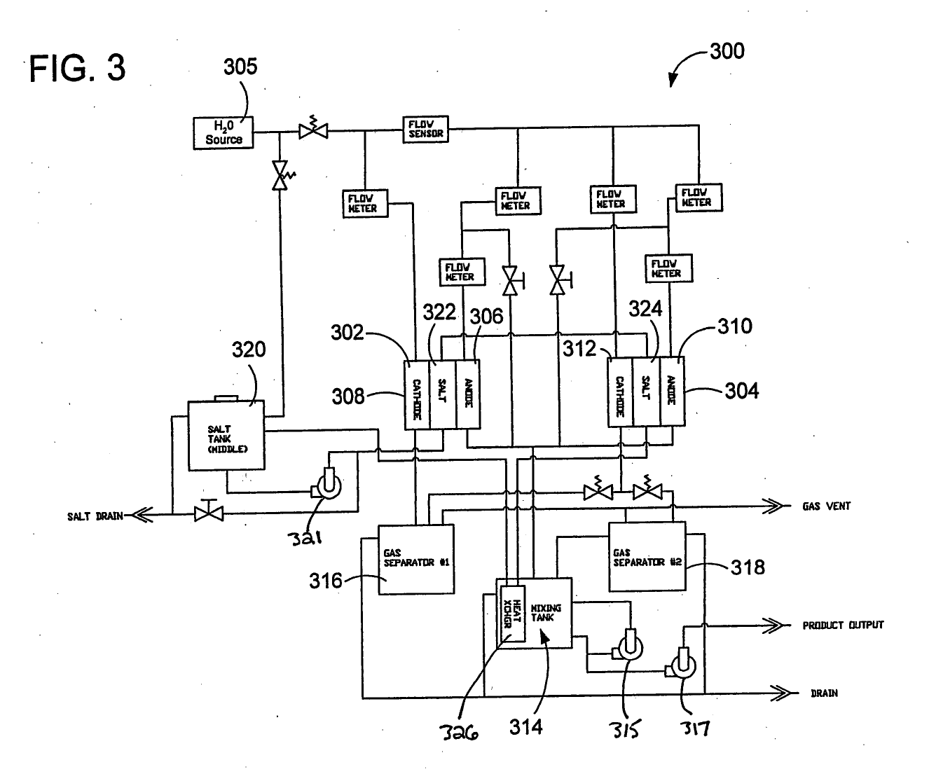

The invention further provides for a process and apparatus for producing an ORP water solution using at least two three chambered electrolysis cells. A diagram of a process for producing an ORP water solution using two electrolysis cells of the invention is shown in FIG. 3.

The process 300 includes two three-chambered electrolytic cells, specifically a first electrolytic cell 302 and second electrolytic cell 304. Water is transferred, pumped or otherwise dispensed from the water source 305 to anode chamber 306 and cathode chamber 308 of the first electrolytic cell 302 and to anode chamber 310 and cathode chamber 312 of the second electrolytic cell 304. Typically, the process of the invention can produce from about 1 liter/minute to about 50 liters/minute of ORP water solution. The production capacity may be increased by using additional electrolytic cells. For example, three, four, five, six, seven, eight, nine, ten or more three-chambered electrolytic cells may be used to in increase the output of the ORP water solution of the invention.

The anode water produced in the anode chamber 306 and anode chamber 310 is collected are collected in the mixing tank 314. A portion of the cathode water produced in the cathode chamber 308 and cathode chamber 312 is collected in mixing tank 314 and combined with the anode water. The remaining portion of cathode water produced in the process is discarded. The cathode water may optionally be subjected to gas separator 316 and/or gas separator 318 prior to addition to the mixing tank 314. The gas separators remove gases such as hydrogen gas that are formed in cathode water during the production process.

The mixing tank 314 may optionally be connected to a recirculation pump 315 to permit homogenous mixing of the anode water and portion of cathode water from electrolysis cells 302 and 304. Further, the mixing tank 314 may optionally include suitable devices for monitoring the level and pH of the ORP water solution. The ORP water solution may be transferred from the mixing tank 314 via pump 317 for application in disinfection or sterilization at or near the location of the mixing tank. Alternatively, the ORP water solution may be dispensed into suitable containers for shipment to a remote site (e.g., warehouse, hospital, etc.).

The process 300 further includes a salt solution recirculation system to provide the salt solution to salt solution chamber 322 of the first electrolytic cell 302 and the salt solution chamber 324 of the second electrolytic cell 304. The salt solution is prepared in the salt tank 320. The salt is transferred via pump 321 to the salt solution chambers 322 and 324. Preferably, the salt solution flows in series through salt solution chamber 322 first followed by salt solution chamber 324. Alternatively, the salt solution may be pumped to both salt solution chambers simultaneously.

Before returning to the salt tank 320, the salt solution may flow through a heat exchanger 326 in the mixing tank 314 to control the temperature of the ORP water solution as needed.

The ions present in the salt solution are depleted over time in the first electrolytic cell 302 and second electrolytic cell 304. An additional source of ions may periodically be added to the mixing tank 320 to replace the ions that are transferred to the anode water and cathode water. The additional source of ions may be used to maintain a constant pH of the salt solution which tends to drop (i.e., become acidic) over time. The source of additional ions may be any suitable compound including, for example, salts such as sodium chloride. Preferably, sodium hydroxide is added to the mixing tank 320 to replace the sodium ions (Na⁺) that are transferred to the anode water and cathode water.

In another embodiment, the invention provides an apparatus for producing an oxidative reductive potential water solution comprising at least two three-chambered electrolytic cells. Each of the electrolytic cells includes an anode chamber, cathode chamber, and salt solution chamber separating the anode and cathode chambers. The apparatus includes a mixing tank for collecting the anode water produced by the electrolytic cells and a portion of the cathode water produced by one or more of the electrolytic cells. Preferably, the apparatus further includes a salt recirculation system to permit recycling of the salt solution supplied to the salt solution chambers of the electrolytic cells.

The following examples further illustrate the invention but, of course, should not be construed as in any way limiting in its scope.

### EXAMPLES 1-3

These examples demonstrate the unique features of the ORP water solution used in the formulation of the invention. The samples of the ORP water solution in Examples 1-3 were analyzed in accordance with the methods described herein to determine the physical properties and levels of ionic and other chemical species present in each sample. The pH, oxidative-reductive potential (ORP) and ionic species present are set forth in Table 1 for each sample of the ORP water solution.

TABLE 1
Physical characteristics and ion species
present for the ORP water solution samples

| | EXAMPLE 1 | EXAMPLE 2 | EXAMPLE 3 |
|---|---|---|---|
| pH | 7.45 | 7.44 | 7.45 |
| ORP (mV) | +879 | +881 | +874 |
| Total Cl⁻ (ppm) | 110 | 110 | 120 |
| Bound Cl⁻ (ppm) | 5 | 6 | 6 |
| Cl Dioxide (ppm) | 1.51 | 1.49 | 1.58 |
| Ozone | 0.12 | 0.10 | 0.12 |
| Hydrogen Peroxide | 42.5 | 43.0 | 42.0 |

As demonstrated by these results, the ORP water solution used in the present invention has suitable physical characteristics for use in disinfection, sterilization and/or cleaning.

### EXAMPLE 4

This example provides a formulation of the invention suitable for topical administration to a patient. The formulation contains the following:

| Component | Quantity | |
|---|---|---|
| ORP water solution | 250 | mL |
| Carbopol ® polymer powder (thickening agent) | 15 | g |
| Triethanolamine (neutralizing agent) | 80 | mL |

### EXAMPLE 5

This example provides a formulation of the invention suitable for topical administration to a patient. The formulation contains the following:

| Component | Quantity | |
|---|---|---|
| ORP water solution | 1000 | mL |
| Carbopol ® polymer powder (thickening agent) | 15 | g |
| Triethanolamine (neutralizing agent) | 80 | mL |

### EXAMPLE 6

This example provides a formulation of the invention suitable for topical administration to a patient. The formulation contains the following:

| Component | Quantity | |
|---|---|---|
| ORP water solution | 250 | mL |
| Carbopol ® polymer powder (thickening agent) | 7 | g |
| Triethanolamine (neutralizing agent) | 12 | mL |

### EXAMPLE 7

This example describes the manufacture of a formulation of the invention comprising an ORP water solution and a thickening agent.

An ORP water solution is put into a suitable container, such as a glass beaker or jar. Carbopol® 974P polymer is passed through a coarse sieve (or strainer), which permits rapid sprinkling, whilst at the same time breaking up any large agglomerates. The polymer Carbopol® 974P is then added as the thickening agent. The Carbopol® polymer is added slowly to prevent the formation of clumps and, thus, avoid an excessively long mixing cycle.

The solution is mixed rapidly during the addition of the Carbopol® polymer so that the powder dissolves at room temperature. The neutralizing agent triethanolamine is then added to the solution and mixed by means of an electric mixer or other suitable device, until a homogeneous gel is obtained. The addition of the neutralizing agent to the Carbopol® polymer composition converts the formulation into a gel.

### EXAMPLE 8

This example describes the use of a gel formulation according to the present invention for the treatment of diabetic foot ulcers.

Six human patients were treated with the gel formulation of Example 6 [confirm this]. All of these patients had major diabetic foot ulcers in the granulating phase. Each wound was cleaned and debribed before treatment. The gel was gently applied to cover the entire area of the would and up to 1 cm outside the wound on the surrounding skin.

The frequency of application of the gel varied according to the nature of each patient's ulcer, with the mean frequency being once every three (3) days. The treatment continued for an average of sixty (60) days. For each of the patients, gross red granulating tissue and enhancement of healthy skin was achieved within 1 to 2 weeks. Accordingly, the gel formulation of the present invention can advantageously be used to treat major diabetic foot ulcers.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A formulation for topical administration comprising an oxidative reductive potential water solution and a thickening agent, wherein the formulation is stable for at least twenty-four hours.

2. The formulation of claim 1, wherein the pH is from about 3 to about 8 and the formulation is stable for at least one week.

3. The formulation of claim 2, wherein the pH is from about 6.4 to about 7.8.

4. The formulation of claim 3, wherein the pH is from about 7.4 to about 7.6.

5. The formulation of claim 3, wherein the formulation is stable for at least two months.

6. The formulation of claim 3, wherein the formulation is stable for at least six months.

7. The formulation of claim 3, wherein the formulation is stable for at least one year.

8. The formulation of claim 3, wherein the formulation is stable for at least three years.

9. The formulation of claim 1, wherein the formulation is selected from the group consisting of a lotion, gel, cream, paste, and ointment.

10. The formulation of claim 9, wherein the formulation is a gel.

11. The formulation of claim 10, wherein the formulation has a viscosity of 10,000 to 100,000 cps.

12. The formulation of claim 10, wherein the thickening agent is present in an amount of from about 1 mg/250 mL of the ORP water solution to about 20 mg/250 mL of the ORP water solution.

13. The formulation of claim 10, wherein the formulation further comprises a neutralizing agent.

14. The formulation of claim 13, wherein neutralizing agent is present in an amount of from about 3% to about 35% by volume, based on the volume of the ORP water solution.

15. A gel for topical administration to a patient comprising an oxidative reductive potential water solution, a thickening agent in an amount of from about 1 mg/250 mL of the ORP water solution to about 20 mg/250 mg of the ORP water solution, and a neutralizing agent in an amount of from about 3% to about 35% by volume based on volume of the ORP water solution, wherein the formulation is stable for at least two months and has pH of about 6.4 to about 7.8.

16. A pharmaceutical dosage form comprising (1) a formulation for topical administration comprising an oxidative reductive potential water solution and a thickening agent and (2) a sealed container, wherein-the formulation is stable for at least twenty-four hours.

17. The dosage form of claim 16, wherein the pH of the formulation is from about 6.4 to about 7.8.

18. The dosage form of claim 17, wherein the pH of the formulation is from about 7.4 to about 7.6.

19. The dosage form of claim 18, wherein the formulation is stable for at least six months.

20. The dosage form of claim 19, wherein the formulation is stable for at least one year.

21. The dosage form of claim 16, wherein the formulation is a lotion, gel, cream, paste, or ointment.

22. The dosage form of claim 21, wherein the formulation is a gel.

23. The dosage form of claim 22, wherein the formulation has a viscosity of 10,000 to 100,000 cps.

24. The dosage form of claim 16, wherein the thickening agent is present in an amount of from about 1 mg/250 mL of the ORP water solution to about 20 mg/250 mL of the ORP water solution.

25. The dosage form of claim 16, wherein the formulation further comprises a neutralizing agent.

26. The dosage form of claim 25, wherein neutralizing agent is present in an amount of from about 3% to about 35% by volume, based on the volume of the ORP water solution.

27. A method for treating a condition in a patient comprising topically administering to a patient a therapeutically effective amount of a formulation comprising an oxidative reductive potential solution and a thickening agent, wherein the formulation is stable for at least about twenty-four hours.

28. The method of claim 27, wherein the formulation is a gel.

29. The method of claim 28, wherein the formulation further comprises a neutralizing agent.

30. A method for promoting wound healing in a patient comprising applying to a wound a formulation comprising an oxidative reductive potential water solution and a thickening agent, wherein the formulation is administered in an amount sufficient to promote wound healing and wherein the formulation is stable for at least about twenty-four hours.

31. The method of claim 30, wherein the formulation is a gel.

32. The method of claim 31, wherein the formulation further comprises a neutralizing agent.

33. A method for preventing a condition in a patient comprising topically administering to a patient a therapeutically effective amount of a formulation comprising an oxidative reductive potential water solution and a thickening agent, wherein the formulation is stable for at least about twenty-four hours.

34. The method of claim 33, wherein the formulation is a gel.

35. The method of claim 34, wherein the formulation further comprises a neutralizing agent.
